(19) United States
(12) Patent Application Publication
(10) Pub. No.: US 2013/0016805 A1
(43) Pub. Date: Jan. 17, 2013

(54) METHOD AND SYSTEM FOR ACQUIRING SPARSE CHANNEL DATA AND FOR IMAGE PROCESSING UTILIZING ITERATIVE RECONSTRUCTION ALGORITHMS

(71) Applicants: Michael D. SILVER, NORTHBROOK, IL (US); Daxin SHI, VERNON HILLS, IL (US)

(72) Inventors: Michael D. SILVER, NORTHBROOK, IL (US); Daxin SHI, VERNON HILLS, IL (US)

(73) Assignees: TOSHIBA MEDICAL SYSTEMS CORPORATION, Otawara-Shi (JP); KABUSHIKI KAISHA TOSHIBA, Tokyo (JP)

(21) Appl. No.: 13/183,867

(22) Filed: Jul. 15, 2011

(51) Int. Cl. A61B 6/03; G01T 1/164

(52) U.S. Cl. 378/4; 250/363.03

(57) ABSTRACT

The current invention is generally related to a data acquisition and or image processing method and system for acquiring and or processing sparse channel data. The sparse channel is implemented in a data acquisition system having a predetermined wider pitch between the adjacent detector cells than that in the currently available imaging systems at least in one predetermined channel direction. The sparse channel is also defined to encompass various imaging modalities including CT, positron emission tomography (PET) and positron emission tomography-computed tomography (PET/CT). The sparse channel data is acquired by the sparse channel data acquisition system, and an image is reconstructed from the sparse channel data according to a predetermined iterative reconstruction technique.

### FIELD OF THE INVENTION

The current invention is generally related to a data acquisition and or image processing method and system for acquiring and or processing sparse channel data.

### BACKGROUND OF THE INVENTION

Currently, commercially available computer tomography (CT) imaging systems are typically equipped with densely installed detector cells along the channel directions. A detector cell means an individual sensor of a two-dimensional array of detectors or detector elements. The detector elements are adjacently installed on a predetermined surface. CT-scanners are geometrically as efficient as possible in closely placing a full complement of the detectors.

In this regard, all of currently practiced reconstruction methods in CT also assume the full complement of densely packed detector channels. These reconstruction methods include filtered backprojection, backprojection filtering and some forms of iterative reconstruction. Current compressive sensing based iterative reconstruction algorithms mainly focus on the sparse view reconstruction based upon projection data that has been acquired from the full complement of densely packed detectors. Compressed sensing total variation (CS-TV) has been shown to give good image quality for sparse views acquired from the full complement of detector channels.

Due to the full complement of dense detector elements, currently available CT imaging systems are expensive. The high costs are substantially due to the above described a large number of detector cells along the channel directions. In addition, an equally large number of necessary electronics units associated with the detector cells also contributes to the expensive costs of these imaging systems. That is, the denser the detector cells are, the more expensive the imaging system becomes. In general, finely pitched detectors of the standard design dramatically increase the hardware costs.

Due to the densely packed detector elements, currently available CT imaging systems also suffer from some undesirable scattering effects. Because of proximity of the adjacent detector elements, X-ray arriving at the detectors are scattered across and over the adjacent detector elements. The densely packed detector elements generally make the scattering correction difficult to achieve a desirable result in reconstructing an artifact-free image.

In view of the above prior art issues, flat panel systems have been considered to reduce the high costs of the standard-design imaging systems. Unfortunately, the flat panel systems generally have lower quantum efficiency and slower readout rates than the standard systems. Quantum efficiency of the flat panel systems is approximately half of the standard systems. The flat panel systems have readout rates of 30 Hz while the standard systems have those of 3000 or more Hz.

In view of the above discussed prior art issues, a practical solution is still desired for a method and a system for reducing the high costs of the detector elements and the associated electronics in the standard CT systems without substantially affecting image quality.

### DESCRIPTION OF THE DRAWINGS

FIG. 1 is a diagram illustrating one embodiment of the multi-slice X-ray CT apparatus or scanner according to the current invention.

FIG. 2A is a diagram illustrating a portion of a prior art configuration having a full complement of detector cells.

FIG. 2B is a diagram illustrating a portion of one embodiment of a sparse-channel X-ray detector having a less-than full complement of detector cells at equidistant according to the current invention.

FIG. 2C is a diagram illustrating a portion of one embodiment of a sparse-channel X-ray detector having a less-than full complement of detector cells at non-equidistant according to the current invention.

FIG. 3A is a diagram illustrating a portion of a prior art two-dimensional configuration having a full complement of detector cells.

FIG. 3B is a diagram illustrating a portion of one embodiment of a sparse-channel X-ray detector having a less-than full complement of detector cells in a first predetermined pattern according to the current invention.

FIG. 3C is a diagram illustrating a portion of one embodiment of a sparse-channel X-ray detector having a less-than full complement of detector cells in a second predetermined pattern according to the current invention.

FIG. 4 is a diagram illustrating a portion of yet another embodiment having a full complement of reconfigurable detector cells according to the current invention.

FIG. 5 is a diagram illustrating an embodiment of the sparse channel detector cells with a collimator according to the current invention.

FIG. 6 is a flow chart illustrating steps involved in a method of acquiring sparse channel data and reconstructing an image based upon the sparse channel data according to the current invention.

FIG. 7A is a reconstructed image of channel data acquired from 892 channels with full view of 900 views per rotation.

FIG. 7B is a reconstructed image of sparse channel data acquired from 224 channels with full view of 900 views per rotation according to the current invention.

FIG. 7C is a reconstructed image of sparse channel data acquired from 112 channels with full view of 900 views per rotation according to the current invention.

### DETAILED DESCRIPTION OF THE PREFERRED EMBODIMENT(S)

In one embodiment of the current invention, the costs of a CT imaging system are substantially reduced by utilizing a fewer number of detector cells than the currently available CT imaging systems in at least in a predetermined channel direction. In general, the costs associated with the detector cells are one of the most expensive parts of the CT imaging systems. In addition, the costs of the electronics units for processing the detected signals from the detector cells are also decreased because a number of the electronics units is reduced for the fewer detector cells.

Assuming that the size of the detector cells remains in an embodiment according to the current invention, as a reduced number of the detector cells is placed on the same surface area of a detector unit, a wider pitch results between at least some pairs of the detector cells. In certain embodiments, the reduced number of the detector cells is installed on the detector unit surface at a predetermined equidistance from each other in one embodiment. Alternatively, the reduced number of the detector cells is installed on the detector unit surface at a predetermined non-uniform distance between the adjacent detector cells in another embodiment.

In either of the above embodiments, because of the reduced number of the detector cells, a pitch is enlarged between at least some pairs of the adjacent detector cells on the detector unit surface for certain functional advantages in addition to the cost advantage. Due to the increased spacing between the adjacent detector cells, the scatter correction procedure is facilitated due to some reduced cross-talk. Furthermore, for some higher spatial resolution embodiments, the quantum efficiency and readout times desirable remain substantially the same as the standard CT systems having a full complement of the detector cells.

In the current application, the term, “sparse channel” or “detector sparse channel” is defined to generally encompass embodiments of the data acquisition system having a predetermined wider pitch between the adjacent detector cells or elements than that in the currently available imaging systems at least in one predetermined channel direction. The sparse channel is also defined to encompass various imaging modalities including CT, positron emission tomography (PET) and positron emission tomography-computed tomography (PET/CT). By the same token, the term, “sparse channel data” is defined to generally encompass data that is acquired by the embodiments of the data acquisition system according to the current invention. Similarly, “sparse-channel projection data” is projection data based upon the sparse channel data.

After projection data is acquired using the above described sparse channel embodiments according to the current invention, the sparse channel data is processed using a compressive sensing based iterative reconstruction algorithm. In prior art, compressed sensing with total variation minimization has been utilized to reconstruct subject information such as x-ray attenuation of a patient as a function of position within the patient based upon sparse view data as acquired by a full complement of detector channels, and the sparseness of views is optionally as low as one tenth of normally available full views. In contrast, one embodiment of the current invention utilizes compressed sensing with total variation minimization to reconstruct subject information within the patient based upon sparse channel data as acquired from the sparse channels but with at least a full density of views. In certain situations, the number of views is optionally increased for sparse channel data according to the current invention.

One exemplary sparse channel data illustrate the following consideration. Assuming that only fan-beam is used for simplicity, when the ordinary number of dense channels is Nch, the channel pitch along the diameter of the field of view (FOV) is FOV/Nch. Assuming further that the periphery of the FOV is πFOV, for the same sampling coverage along the πFOV, it is approximated that πFOV/Nviews=FOV/Nch, where Nviews is the number of views per rotation. This suggests Nviews=πCNch. On the other hand, standard CT systems are designed to have approximately Nch=Nviews=1000. In order to achieve clinically acceptable image quality using compressed sensing total variation, a greater number of views above 1000 is necessary for ⅛ sparse channel data that has been acquired from an embodiment having approximately one eighth of the full complement of the detector cells. To further illustrate the above example of the sparse channels and the sparse channel data with a compressed sensing total variation reconstructive technique, a full complement of 1024 detector cells is reduced to 128 detector cells while the number of views per rotation is optionally increased to about 3000.

As the standard CT detector technology improves, the sensor size has become several times smaller than the past detector size. The improved smaller standard detectors are advantageous over flat panel detectors for implementing the embodiments of the sparse channel data acquisition system according to the current invention. Although flat panel detectors have the required spatial resolution, they have substantially low quantum efficiency and long readout times. On the other hand, the standard CT detectors are substantially high quantum efficiency and short readout times. Because of the smaller size, if the same number of smaller detector cells is installed on the same detector surface in the data acquisition system, an embodiment of the sparse channel is implemented for the current invention. By the same token, since electronic packaging has equally improved in size, an anti-scatter collimator becomes easier to align. On the other hand, since a larger number of views per rotation is optionally necessary for the sparse channel data, unmeasured dose may increase to an patient. To substantially avoid the increased dosage to the patient, an array of pinhole collimators is placed on the X-ray source.

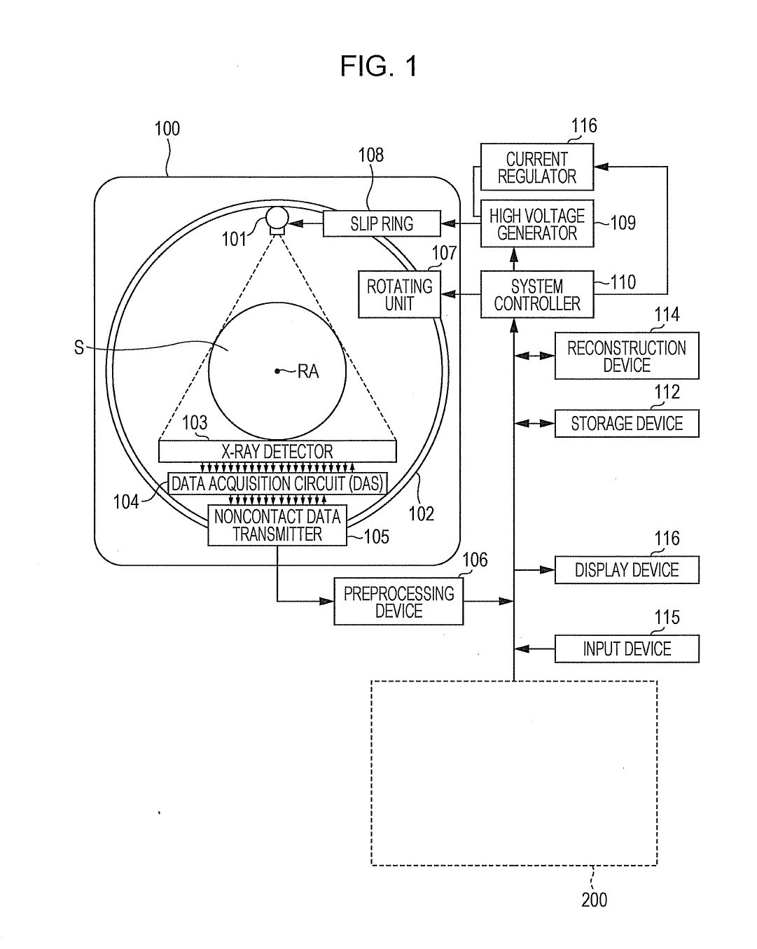

Referring now to the drawings, wherein like reference numerals designate corresponding structures throughout the views, and referring in particular to FIG. 1, a diagram illustrates one embodiment of the multi-slice X-ray CT apparatus or scanner according to the current invention including a gantry 100 and other devices or units. The gantry 100 is illustrated from a front view and further includes an X-ray tube 101, an annular frame 102 and a multi-row or two-dimensional array type X-ray detector 103. The X-ray tube 101 and X-ray detector 103 are diametrically mounted across a subject S on the annular frame 102, which rotates around axis RA. A rotating unit 107 rotates the frame 102 at a high speed such as 0.4 sec/rotation while the subject S is being moved along the axis RA into or out of the illustrated page.

The multi-slice X-ray CT apparatus further includes a high voltage generator 109 that applies a tube voltage to the X-ray tube 101 so that the X-ray tube 101 generates X ray. In one embodiment, the high voltage generator 109 is mounted on the frame 102. The X rays are emitted towards the subject S, whose cross sectional area is represented by a circle. The X-ray detector 103 is located at an opposite side from the X-ray tube 101 across the subject S for detecting the emitted X rays that have transmitted through the subject S.

Still referring to FIG. 1, the X-ray CT apparatus or scanner further includes other devices for processing the detected signals from X-ray detector 103. A data acquisition circuit or a Data Acquisition System (DAS) 104 converts a signal output from the X-ray detector 103 for each channel into a voltage signal, amplifies it, and further converts it into a digital signal. The X-ray detector 103 and the DAS 104 are configured to handle a predetermined total number of projections per rotation (TPPR).

The above described data is sent to a preprocessing device 106, which is housed in a console outside the gantry 100 through a non-contact data transmitter 105. The preprocessing device 106 performs certain corrections such as sensitivity correction on the raw data. A storage device 112 then stores the resultant data that is also called projection data at a stage immediately before reconstruction processing. The storage device 112 is connected to a system controller 110 through a data/control bus, together with a reconstruction device 114, display device 116, input device 115, and the scan plan support apparatus 200. The scan plan support apparatus 200 includes a function for supporting an imaging technician to develop a scan plan.

One embodiment of the reconstruction device 114 includes various software and hardware components. According to one aspect of the current invention, the X-ray detector 103 of the CT apparatus is advantageously configured to implement the detector sparse channels having a predetermined increased pitch between the detector cells or elements on a surface of the X-ray detector 103 in one embodiment. According to the current invention, the embodiment of the X-ray detector 103 has a certain predetermined range in a total number of the detector cells, and the predetermined range is generally between one tenth and one fourth of a full complement of the currently available or conventional prior art X-ray detector cells. In embodiments of the current invention, an exemplary number of the detector cells is as small as one eighth of the full complement of the prior art detector cells.

According to another aspect of the current invention, the reconstruction device 114 of the CT apparatus advantageously minimizes total variation (TV) using an iterative reconstruction technique. In general, the reconstruction device 114 in one embodiment of the current invention performs on the sparse channel data the total variation iterative reconstruction (TVIR) algorithm for reconstructing an image. In further detail, one embodiment of the reconstruction device 114 performs on the sparse channel data an ordered subset simultaneous algebraic reconstruction technique (OSSART) process and a TV minimization process.

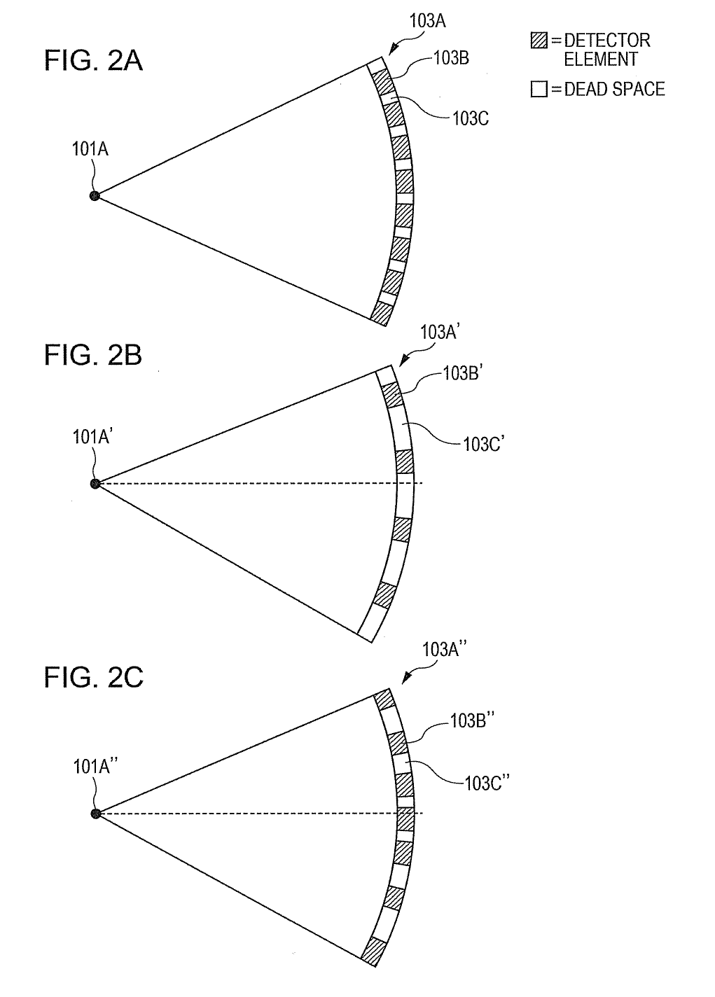

Now referring to FIGS. 2A, 2B and 2C, diagrams illustrate a comparison among detector cell configurations in one dimension according to the current invention and the prior art. With respect to the diagrams, an effective area or space of a detector cell is an area where a single detector cell occupies while a dead area or space is an area where no detector cell occupies. For the purpose of this application, the effective area or space is considered substantially the same as the physical size of a detector cell, which is indicated by a dark box.

FIG. 2A is a diagram illustrating a portion of a prior art configuration having a full complement of detector cells or detector elements. An X-ray source 101A is located opposite an X-ray detector 103A across a region of interest to be imaged by X rays. In the prior art configuration, the detector cells 103B are indicated by a row of equal-size dark boxes along one channel direction or one dimension. Between the detector cells 103B, dead spaces 103C are indicated by a row of light boxes also along the same channel direction or one dimension. The dead spaces 103C are empty spaces that are not occupied by the detector cells 103B. A pitch is defined to be a distance between two centers of the adjacent detector cells 103B. The diagram may be considered as a cross sectional view of the X-ray detector 103A. The diagram also may be considered to indicate a fan beam that is emitted from the X-ray source 101A. Alternatively, the diagram illustrates a one-dimensional array detector 103A.

FIG. 2B is a diagram illustrating a portion of one embodiment of a sparse-channel X-ray detector 103A′ having a less-than full complement of detector cells or detector elements 103B′. An X-ray source 101A′ is located opposite the X-ray detector 103A′ across a region of interest to be imaged by X rays. In an exemplary embodiment, the detector cells 103B′ are indicated by a row of equal-size dark boxes along one channel direction or one dimension. Between the detector cells 103B′, dead spaces 103C′ are indicated by a row of light boxes also along the same channel direction or one dimension. The dead spaces 103C′ are empty spaces that are not occupied by the detector cells 103B′. A pitch is defined to be a distance between two centers of the adjacent detector cells 103B′. The pitch of every pair of the detector cells 103B′ is larger than that of the detector cells 103B of the prior art detector 103A in FIG. 2A.

Still referring to FIG. 2B, the detector cells 103B′ are placed at a predetermined equidistance with each other. In other words, the increased pitch is the same between any pair of the adjacent detector cells 103B′ across the X-ray detector 103A′. The diagram may be considered as a cross sectional view of the X-ray detector 103A′. The diagram also may be considered to indicate a fan beam that is emitted from the X-ray source 101A′. Alternatively, the diagram illustrates a one-dimensional array detector 103A′.

Lastly, the detector cells 103B′ are asymmetrical about the dotted central line in one embodiment of the X-ray detector 103A′ according to the current invention. The detector cells 103B′ are shifted or rotated by one quarter pixel or a predetermined angle in a counter-clock direction. The quarter-offset design is known in the CT scanners in order to promote the fill-in effect so as to improve the sampling density level while the X-ray detector 103A′ rotates.

FIG. 2C is a diagram illustrating a portion of another embodiment of a sparse-channel X-ray detector 103A″ having a less-than full complement of detector cells 103B″. An X-ray source 101A″ is located opposite the X-ray detector 103A″ across a region of interest to be imaged by X rays. In an exemplary embodiment, the detector cells 103B″ are indicated by a row of equal-size dark boxes along one channel direction or one dimension. Between the detector cells 103B″, dead spaces 103C″ are indicated by a row of light boxes also along the same channel direction or one dimension. The dead spaces 103C″ are empty spaces that are not occupied by the detector cells 103B″. A pitch is defined to be a distance between two centers of the adjacent detector cells 103B″. The pitch of at least some of the detector cells 103B″ is larger than that of the detector cells 103B of the prior art detector 103A in FIG. 2A.

Still referring to FIG. 2C, the detector cells 103B″ are placed at a predetermined distance with each other. In other words, the increased pitch is not necessarily the same among pairs of the adjacent detector cells 103B″ across the X-ray detector 103A′. In the illustrated embodiment, the pitch increases from the central region toward the peripheral region. In alternative embodiment, the pitch in peripheral regions is larger than that in central regions without gradual increase. The diagram may be considered as a cross sectional view of the X-ray detector 103A″. The diagram also may be considered to indicate a fan beam that is emitted from the X-ray source 101A″. Alternatively, the diagram illustrates a one-dimensional array detector 103A″.

Lastly, the detector cells 103B″ are asymmetrical about the dotted central line in one embodiment of the X-ray detector 103A″ according to the current invention. The detector cells 103B″ are shifted or rotated by one quarter pixel or a predetermined angle in a counter-clock direction. The quarter-offset design is known in the CT scanners in order to promote the fill-in effect so as to improve the sampling density level while the X-ray detector 103A″ rotates.

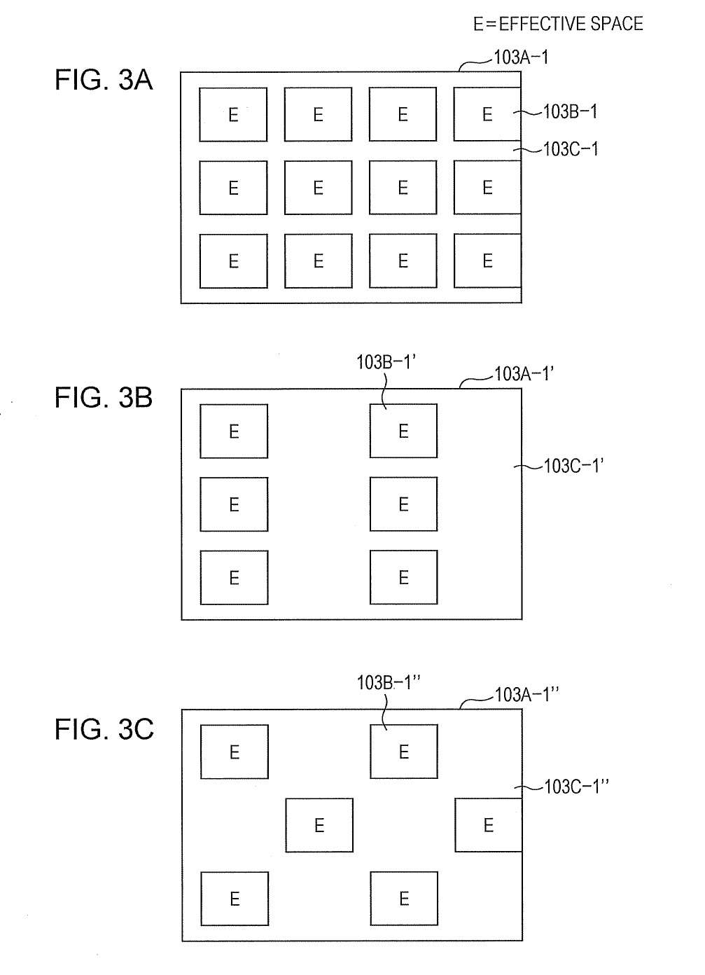

Now referring to FIGS. 3A, 3B and 3C, diagrams illustrate a comparison among detector cell configurations in two dimensions according to the current invention and the prior art. With respect to the diagrams, an effective area or space of a detector cell is an area where a single detector cell occupies while a dead area or space is an area where no detector cell occupies. For the purpose of this application, the effective area or space is considered substantially the same as the physical size of a detector cell, which is indicated by a box E.

FIG. 3A is a diagram illustrating a portion of a prior art configuration having a full complement of detector cells. In the prior art configuration, the detector cells 103B-1 are indicated by rows and columns of equal-size boxes along two channel directions or two dimensions. Between the detector cells 103B-1, dead spaces 103C-1 are indicated by rows and columns of light areas also along the same channel directions or two dimensions. The dead spaces 103C-1 are empty spaces that are not occupied by the detector cells 103B-1. A pitch is defined to be a distance between two centers of the adjacent detector cells 103B-1, and the pitch are optionally either equal or different between the two channel directions.

FIG. 3B is a diagram illustrating a portion of one embodiment of a sparse-channel X-ray detector 103A-1′ having a less-than full complement of detector cells 103B-1′. An X-ray source is not illustrated, but it is located opposite the X-ray detector 103A-1′ across a region of interest to be imaged by X rays. In an exemplary embodiment, the detector cells 103B-1′ are indicated by rows and columns of equal-size boxes along the two channel directions or two dimensions. Between the detector cells 103B-1′, dead spaces 103C-1′ are indicated by rows and columns of areas also along the same two channel directions or two dimensions. The dead spaces 103C-1′ are empty spaces that are not occupied by the detector cells 103B-1′. A pitch is defined to be a distance between two centers of the adjacent detector cells 103B-1′. The pitch of every pair of the detector cells 103B-1′ is larger at least in one channel direction than that of the detector cells 103B of the prior art detector 103A in FIG. 2A.

Still referring to FIG. 3B, the detector cells 103B-1′ are placed at a predetermined equidistance with each other. In other words, the pitch is the same between any pair of the adjacent detector cells 103B-1′ in either horizontal or vertical direction across the X-ray detector 103A-1′. However, in comparison to the prior art configuration as shown in FIG. 3A, the horizontal pitch is increased while the vertical pitch remains the same in the embodiment according to the current invention. The increased horizontal pitch implements the sparse-channel X-ray detector 103A-1′ having a less-than full complement of detector cells 103B-1′ and an appearance of stripes.

FIG. 3C is a diagram illustrating a portion of another embodiment of a sparse-channel X-ray detector 103A-1″ having a less-than full complement of detector cells 103B-1″. An X-ray source is not illustrated, but it is located opposite the X-ray detector 103A-1″ across a region of interest to be imaged by X rays. In an exemplary embodiment, the detector cells 103B-1″ are indicated by rows and columns of equal-size boxes along the two channel directions or two dimensions. Between the detector cells 103B-1″, dead spaces 103C-1″ are indicated by spots of areas also along the same two channel directions or two dimensions. The dead spaces 103C-1″ are empty spaces that are not occupied by the detector cells 103B-1″. A pitch is defined to be a distance between two centers of the adjacent detector cells 103B-1″. The pitch of every pair of the detector cells 103B-1″ is larger in both channel directions than that of the detector cells 103B of the prior art detector 103A in FIG. 2A.

Still referring to FIG. 3C, the detector cells 103B-1″ are placed at a predetermined equidistance with each other. In other words, the pitch is the same between any pair of the adjacent detector cells 103B-1′ in both horizontal and vertical directions across the X-ray detector 103A-1″. However, in comparison to the prior art configuration as shown in FIG. 3A, both the horizontal pitch and the vertical pitch are increased by substantially the same distance in the embodiment according to the current invention. The increased horizontal and vertical pitches implement the sparse-channel X-ray detector 103A-1″ having a less-than full complement of detector cells 103B-1″ and an appearance of a checkerboard.

In alternative embodiments, the vertical pitch and or horizontal pitch are optionally non-equidistant. In other words, a combination of the vertical pitch and the horizontal pitch of the detector cells is optionally increasing or decreasing across the surface of the detector. For example, the vertical pitch and or the horizontal pitch of the detector cells increase towards peripheral areas of the detector as illustrated in one-dimensional array of detector cells in FIG. 2C.

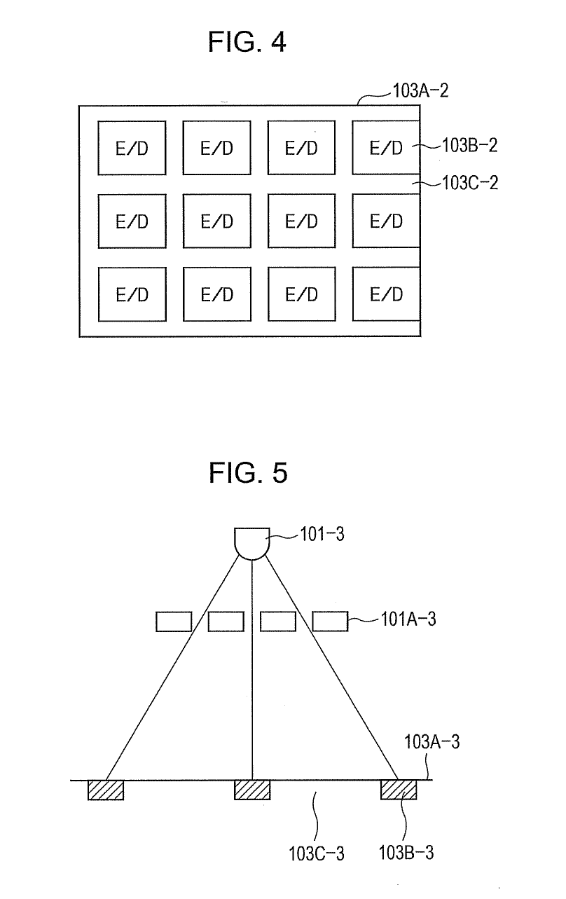

Now referring to FIG. 4, a diagram illustrates a portion of yet another embodiment having a full complement of reconfigurable detector cells 103B-2. In the configuration, the detector cells 103B-2 are indicated by rows and columns of equal-size boxes along two channel directions or two dimensions. Between the detector cells 103B-2, dead spaces 103C-2 are indicated by rows and columns of light areas also along the same channel directions or two dimensions. The dead spaces 103C-2 are empty spaces that permanently lack the detector cells 103B-2. In addition, the dead spaces 103C-2 also include areas that are occupied by the detector cell 103B-2, which is not activated or is deactivated. A pitch is defined to be a distance between two centers of the adjacently located and activated detector cells 103B-2, and the pitch are optionally either equal or different between the two channel directions.

Still referring to FIG. 4, the diagram illustrates one embodiment of an adjustable sparse-channel X-ray detector 103A-2 optionally having a less-than full complement of active detector cells 103B-2. An X-ray source is not illustrated, but it is located opposite the X-ray detector 103A-2 across a region of interest to be imaged by X rays. In an exemplary embodiment, the detector cells 103B-2 are each indicated by rows and columns of equal-size boxes E/D along the two channel directions or two dimensions. For the purpose of the current specification, an effective area or space E of a detector cell is an area where a single active detector cell occupies, and the effective area or space is considered substantially the same as the physical size of an active detector cell 103B-2. On the other hand, when a particular set of the detector cells 103B-2 is not activated or deactivated, these inactive detector cells 103B-2 functionally become a dead area D and a part of the dead spaces 103C-2. Since a pitch is defined to be an adjustable distance between two centers of the adjacent active detector cells 103B-2, the pitch of a particular pair of the detector cells 103B-2 is optionally adjusted to be larger than that of the detector cells 103B-2 of the prior art detector 103A in FIG. 2A. The adjustable pitch is related to the total number of active detector cells 103B-2, which ranges from one tenth to one fourth of a full complement of the detector cells 103B of the prior art detector 103A in FIG. 2A. The pitch adjustment is generally made via a control mechanism prior to scanning based upon a certain application. The adjustable horizontal and or vertical pitches implement the reconfigurable sparse-channel X-ray detector 103A-2 optionally having a less-than full complement of detector cells 103B-2.

In the above described embodiments of the data acquisition system according to the current invention, the sparse channel data is obtained for reconstruction and optionally has a set of full views. In one example, the sparse channel full view data generally ranges from 900 views to 1200 views per rotation. The above described embodiments of the data acquisition system according to the current invention also optionally acquires sparse channel data having a set of sparse views for reconstruction. In one example, a set of sparse view data has less than 900 views per rotation. For reconstructing an image from the sparse channel data, a reconstruction algorithm is iterative such as the total variation iterative reconstruction (TVIR) algorithm. In further detail, one embodiment of the reconstruction device 114 performs on the sparse channel data an ordered subset simultaneous algebraic reconstruction technique (OSSART) process and a TV minimization process. If the sparse channel data has less than full views, a number of iterations in the above iterative reconstruction algorithm optionally increases.

Now referring to FIG. 5, a diagram illustrates an embodiment of the sparse channel detector cells with a collimator according to the current invention. A partial diagram of the exemplary embodiment of a sparse-channel X-ray detector 103A′-3 has a less-than full complement of detector cells 103B-3. An X-ray source 101-3 is located opposite the X-ray detector 103A-3 across a region of interest to be imaged by X rays. In the exemplary embodiment, the detector cells 103B-3 are indicated by a row of equal-size dark boxes along one channel direction or one dimension. Between the detector cells 103B-3, dead space 103C-3 is indicated by a light color also along the same channel direction or one dimension. The dead spaces 103C-3 are empty spaces that are not occupied by the detector cells 103B-3. A pitch is defined to be a distance between two centers of the adjacent detector cells 103B-3. The pitch of every pair of the detector cells 103B-3 is larger than that of the detector cells 103B of the prior art detector 103A in FIG. 2A.

The exemplary embodiment of FIG. 5 has a collimator 101 A-3 that is placed near the X-ray source 101-3 for limiting the X-rays emitted from the X-ray source 101 towards the X-ray detector 103A-3. The collimator 101A-3 is designed to block X-rays that are not ultimately reaching the detector cells 103B-3 while allowing some X-rays that are ultimately reaching the detector cells 103B-3. Although the collimator 101A-3 may not be perfect in selecting an exact direction of the X-rays, the collimator 101A-3 substantially lowers the X-ray dosage to a patient and allows a sufficient amount of the X-rays to be detected at the detector cells 103B-3.

Still referring to FIG. 5, the detector cells 103B-3 are placed at a predetermined equidistance with each other. In other words, the increased pitch is the same between any pair of the adjacent detector cells 103B-3 across the X-ray detector 103A-3 in the exemplary embodiment. The diagram may be considered as a cross sectional view of the X-ray detector 103A-3. The diagram also may be considered to indicate a fan beam that is emitted from the X-ray source 101-3. Alternatively, the diagram illustrates a one-dimensional array detector 103A-3. In other embodiments, embodiments have two-dimensional array detector 103A-3 according to the current invention. Furthermore, other embodiments according to the current invention have the detector cells 103B-3 that are placed at a predetermined non-equidistance with each other, and the non-equidistance increases or decreases depending on the relative position of the detector cells 103B-3 in the X-ray detector 103A-3.

In still other embodiments, a combination of the above described features is provided to practice the current invention. For example, a particular one of the predetermined collimators 101A-3 is placed near the X-ray source 101-3 for limiting the X-rays emitted from the X-ray source 101 towards a corresponding pattern of the detector cells 103B-3. In other words, the collimators 101A-3 have through holes that are equidistant or non-equidistant in certain predetermined patterns such as stripes and checkerboard. The collimators 101A-3 are optionally designed to be even reconfigurable in certain embodiments.

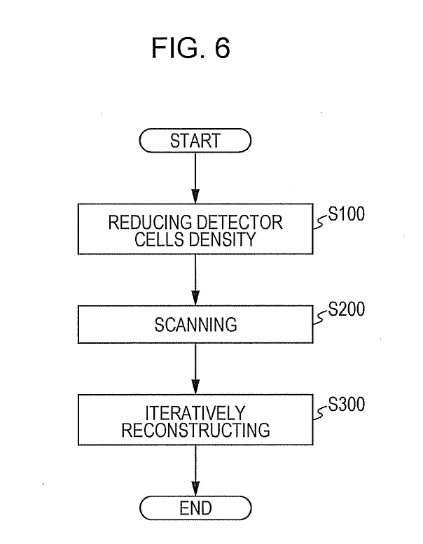

Now referring to FIG. 6, a flow chart illustrates steps involved in a method of acquiring sparse channel data and reconstructing an image based upon the sparse channel data according to the current invention. In a step S 100, the X-ray detector of the CT apparatus is advantageously configured to implement the sparse channels having a predetermined increased pitch between the detector cells on a surface of the X-ray detector according to one method of the current invention. According to the current invention, the embodiment of the X-ray detector has a certain predetermined range in a total number of the detector cells, and the predetermined range is generally between one tenth and one fourth of a full complement of the currently available or conventional prior art X-ray detector cells. In embodiments of the current invention, an exemplary number of the detector cells is as small as one eighth of the full complement of the prior art detector cells. Furthermore, in the step S100, the X-ray detector of the CT apparatus is advantageously configured to implement the sparse channels in a predetermined pitch and pattern such as stripes and checkerboard in a dimensional detector. The pitch is optionally equidistant or non-equidistant among the detector cells. The step S100 is optionally implemented in a scan plan support apparatus for supporting an imaging technician to develop a scan plan.

In a step S200 of the exemplary method of acquiring sparse channel data according to the current invention, a region of interest is scanned. The X rays are emitted from an X-ray tube towards a subject. The X-ray detector is located at an opposite side from the X-ray tube across the subject and includes sparse channel detector cells for detecting the emitted X rays that have transmitted through the region of interest in the subject. In the step S200, the X-ray CT apparatus or scanner further includes other devices for processing the detected signals from X-ray detector having sparse channels. A data acquisition circuit or a Data Acquisition System (DAS) converts a signal output from the X-ray detector for each channel into a voltage signal, amplifies it, and further converts it into a digital signal. The X-ray detector and the DAS are configured to handle a predetermined total number of projections per rotation (TPPR).

Still in the step S200, the above described sparse channel data is sent to a preprocessing device performing certain corrections such as sensitivity correction on the raw data. A storage device then stores the resultant sparse channel data that is also called projection data at a stage immediately before reconstruction processing.

Still referring to FIG. 6, in a step S300 of the exemplary method of acquiring sparse channel data and reconstructing an image according to the current invention, a reconstruction device of the CT apparatus advantageously minimizes total variation (TV) using an iterative reconstruction technique. In general, the reconstruction device in one embodiment of the current invention performs on the sparse channel data the total variation iterative reconstruction (TVIR) algorithm for reconstructing an image. In further detail, one embodiment of the reconstruction device 114 performs on the sparse channel data an ordered subset simultaneous algebraic reconstruction technique (OSSART) process and a TV minimization process. The variations of the above mentioned iterative reconstruction techniques further include other algebraic reconstruction techniques (ART) and Expectation-Maximization (EM) reconstruction techniques. If the sparse channel data has less than full views, a number of iterations in the above iterative reconstruction algorithm optionally increases.

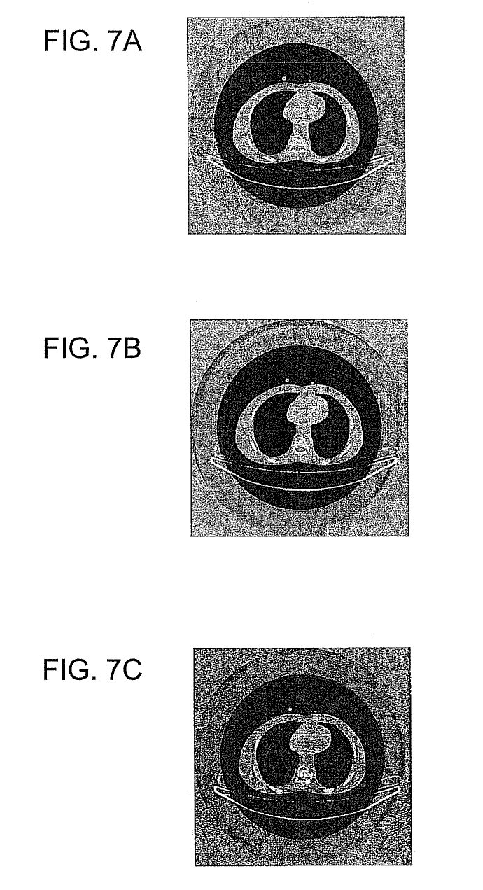

FIGS. 7A, 7B and 7C illustrate the resultant image comparison of the sparse channel data according to the current invention. The number of channels is varies while the number of views is maintained during the data acquisition. In general, the image quality in the reconstructed images in FIGS. 7A, 7B and 7C is clinically acceptable.

FIG. 7A is a reconstructed image of channel data acquired from 892 channels with full view of 900 views per rotation. The resultant image is reconstructed from the above projection data using a predetermined iterative reconstruction with total variation minimization (IRTV) after 100 iterations. The general image quality of the resultant image is clinically acceptable.

FIG. 7B is a reconstructed image of sparse channel data acquired from 224 channels with full view of 900 views per rotation. The sparse channel is one fourth of the channels in FIG. 7A. The resultant image is reconstructed from the above projection data using a predetermined iterative reconstruction with total variation minimization (IRTV) after 100 iterations. The general image quality of the resultant image is clinically acceptable.

FIG. 7C is a reconstructed image of sparse channel data acquired from 112 channels with full view of 900 views per rotation. The sparse channel is one eighth of the channels in FIG. 7A. The resultant image is reconstructed from the above projection data using a predetermined iterative reconstruction with total variation minimization (IRTV) after 500 iterations. The number of iterations is optionally increased five times from the iterations in FIG. 7A. The general image quality of the resultant image is clinically acceptable.

It is to be understood, however, that even though numerous characteristics and advantages of the present invention have been set forth in the foregoing description, together with details of the structure and function of the invention, the disclosure is illustrative only, and that although changes may be made in detail, especially in matters of shape, size and arrangement of parts, as well as implementation in software, hardware, or a combination of both, the changes are within the principles of the invention to the full extent indicated by the broad general meaning of the terms in which the appended claims are expressed.

What is claimed is:

1. An imaging system, comprising:
an X-ray source for emitting X-rays towards an object;
detector elements configured to have a pitch between said detector elements at least in a channel direction for detecting the X-rays representing a portion of the object, the predetermined pitch being larger than a conventional pitch so as to realize sparse detector channels;
electronics units each associated with corresponding one of the sparse detector channels for generating sparse-channel projection data; and
a processing unit connected to said electronics units for reconstructing an image from the sparse-channel projection data using an iterative reconstruction algorithm.

2. The imaging system according to claim 1 wherein said detector elements are placed on a two-dimensional plane in a predetermined pattern.

3. The imaging system according to claim 2 wherein the predetermined pattern is a striped pattern.

4. The imaging system according to claim 2 wherein the predetermined pattern is a checker-board pattern.

5. The imaging system according to claim 1 wherein the detector elements are reconfigurable.

6. The imaging system according to claim 5 further comprising a collimator placed near said X-ray source for adjusting the X-rays.

7. The imaging system according to claim 1 wherein said detector elements have a detector sparseness ranging from ¼ to 1/10 of a conventional number of said detector elements.

8. The imaging system according to claim 7 wherein said detector elements have a detector sparseness of 1/8.

9. The imaging system according to claim 1 wherein the iterative reconstruction algorithm includes one of ART, TV, SART with TV and EM.

10. The imaging system according to claim 1 wherein said processing unit performs a larger number of iterations for the sparse-channel projection data from the detector elements.

11. The imaging system according to claim 1 wherein the sparse-channel projection data has at least a full complement of views.

12. The imaging system according to claim 1 wherein the sparse-channel projection data has less than a full complement of views.

13. The imaging system according to claim 1 wherein the imaging system includes a group of modalities comprising computed tomography (CT), positron emission tomography (PET) and positron emission tomography-computed tomography (PET/CT).

14. A method of imaging, comprising the steps of:
emitting X-rays from an X-ray source towards an object;
providing detector elements configured to have a pitch between the detector elements at least in a channel direction for detecting the X-rays representing a portion of the object, the predetermined pitch being larger than a conventional pitch so as to realize sparse detector channels;
providing electronics units each associated with corresponding one of the sparse detector channels for generating sparse-channel projection data; and
reconstructing an image from the sparse-channel projection data using an iterative reconstruction algorithm.

15. The method of imaging according to claim 14 wherein the detector elements are placed on a two-dimensional plane in a predetermined pattern.

16. The method of imaging according to claim 15 wherein the predetermined pattern is a striped pattern.

17. The method of imaging according to claim 15 wherein the predetermined pattern is a checker-board pattern.

18. The method of imaging according to claim 14 wherein the detector elements are reconfigurable.

19. The method of imaging according to claim 18 further comprising an additional step of adjusting the X-rays via a collimator placed near the X-ray source.

20. The method of imaging according to claim 14 wherein the detector elements have a detector sparseness ranging from ¼ to 1/10 of a conventional number of the detector elements.

21. The method of imaging according to claim 20 wherein the detector elements have a detector sparseness of 1/8.

22. The method of imaging according to claim 14 wherein the iterative reconstruction algorithm includes one of ART, TV, SART with TV and EM.

23. The method of imaging according to claim 14 wherein a larger number of iterations is performed for the sparse-channel projection data from the detector elements having

24. The method of imaging according to claim 14 wherein the sparse-channel projection data has at least a full complement of views.

25. The method of imaging according to claim 14 wherein the sparse-channel projection data has less than a full complement of views.

26. The method of imaging according to claim 14 wherein the imaging method is applicable to a group of modalities comprising computed tomography (CT), positron emission tomography (PET) and positron emission tomography-computed tomography (PET/CT).
